SEQUENCE LISTING (6 sequences)

SEQ ID NO: 1 - DNA, 221 bp, Artificial Sequence
  Description of Artificial Sequence Synthetic oligonucleotide
ggggaccccccggaaggggggacgaggtgcgggcacctcgtacgggagttcgaccgtgacgagtcacgggctagcgctttcgcgctctcccaggtgacgcctcgtgaagaggcgcgaccttcgtgcgtttcggcgacgcacgagaaccgccacgctgcttcgcagcgtggccccttcgcgcagcccgctgcgcgaggtgacccccgaaggggggttcccca

SEQ ID NO: 2 - DNA, 86 bp, Artificial Sequence
  Description of Artificial Sequence Synthetic oligonucleotide
gggttcatagcctattcggcttttaaaggacctttttccctcgcgtagctagctacgcgaggtgaccccccgaaggggggtgcccc

SEQ ID NO: 3 - DNA, 130 bp, Artificial Sequence
  Description of Artificial Sequence Synthetic oligonucleotide
gggttcatagcctattcggcttcgcgcatgggaatttgagggacgatggggaagtgggagcgcgttttaaaggacctttttccctcgcgtagctagctacgcgaggtgaccccccgaaggggggtgcccc

SEQ ID NO: 4 - DNA, 118 bp, Artificial Sequence
  Description of Artificial Sequence Synthetic oligonucleotide
gggttcatagcctattcggcttcgcgcccgtttataatacttagtgagcgcgttttaaaggacctttttccctcgcgtagctagctacgcgaggtgaccccccgaaggggggtgcccc

SEQ ID NO: 5 - DNA, 118 bp, Artificial Sequence
  Description of Artificial Sequence Synthetic oligonucleotide
gggttcatagcctattcggcttcgcgcccctggggtttgcctcaggagcgcgttttaaaggacctttttcccttgcgtagctagctacgcgaggtgaccccccgaaggggggtgcccc

SEQ ID NO: 6 - RNA, 196 nt, Artificial Sequence
  Description of Artificial Sequence Synthetic oligonucleotide
gggguuuccaaccggaauuugagggaugccuaggcaucccccgugcgucccuuuacgagggauugucgacucuagucgacgucugggcgaaaaauguacgagaggaccuuuucgguacagacgguaccugagggaugccuaggcaucccccgcgccgguuucggaccuccagugcguguuaccgcacugucgaccc

(19) United States
(12) Patent Application Publication
(10) Pub. No.: US 2005/0214805 A1
(43) Pub. Date: Sep. 29, 2005

(54) Methods of detection employing immuno-Q-Amp technology

(75) Inventor: Abraham Grossman, New York, NY (US)

Correspondence Address: DAVIDSON, DAVIDSON & KAPPEL, LLC, 485 SEVENTH AVENUE, 14TH FLOOR, NEW YORK, NY, 10018, US

(73) Assignee: Q-RNA, Inc., New York, NY (US)

(21) Appl. No.: 10/985,183

(22) Filed: Nov. 9, 2004

Related U.S. Application Data
(60) Provisional application No. 60/519,035, filed on Nov. 10, 2003.

(51) Int. Cl. C12Q 1/68; G01N 33/53; G01N 33/567

(52) U.S. Cl. 435/6; 435/7.2

(57) ABSTRACT

Disclosed herein are methods of detecting target molecules from a complex sample using a modified affinity ligand. In particular, the modified affinity ligand is an antibody specific for the target and has conjugated to it a nucleic acid molecule. This conjugated nucleic acid molecule serves as a template for a replicase that can replicate the template a billion-fold in an matter of minutes. The replication products then serve to facilitate a detectable signal indicating the presence of the target in the sample.

### RELATED APPLICATIONS

This application claims the benefit of and priority to U.S. Provisional application 60/519,035, filed Nov. 10, 2003.

### FIELD OF THE INVENTION

The present invention relates to methods of detecting target molecules using modified detector molecules wherein the modification facilitates signal amplification.

### BACKGROUND OF THE INVENTION

It is often desirable to detect the presence or absence of one or more relevant molecules in a complex sample. For example, it may be important to detect changes in a molecule that was subjected to certain conditions. Proteins can undergo conformational changes under certain conditions. These conformational changes can be important diagnostically providing an opportunity for early detection. Detecting such conformationally altered proteins can assist in the treatment of a particular disease or syndrome.

Some disease processes appear to stem from the misfolding of proteins. This misfolding is often associated with nucleic acids (NAs) and cellular factors. These misfolded proteins can go on to form pathological agglomerations. These agglomerations have been shown to be associated with neuronal cell death and brain wasting diseases such as Alzheimer's and Parkinson's disease in humans, scrapie, mad cow disease and chronic wasting diseases in animals. Spongiform encephalopathies, often involved with certain neuronal cell death and brain wasting syndromes, characteristically have protein plaques or agglomerations made manifest upon dissection. In spongiform encephalopathies, prion proteins are thought to be the etiologic agent. Prion-based diseases result from “infectious proteins” that are cellular benign prion proteins misfolded into an infectious isoform. This infectious isoform is involved in pathological protein agglomeration. Misfolded proteins also appear to damage cells in the lung, heart, kidney, pancreas and other organs.

There is a need to detect molecules in a sample that may indicate a pathological process. The present invention addresses this need.

### BRIEF SUMMARY OF THE INVENTION

The present invention pertains to methods of detecting molecules from a complex sample using a modified detector molecule. In particular, the modified detector molecule is specific for a particular target molecule and comprises a template that can be employed to amplify a signal indicating the presence of the target molecule.

One embodiment of the invention is directed to a method for detecting the presence or absence of a target molecule. The methods of the present invention comprise a detector molecule (or affinity ligand) having affinity for a target molecule. In one aspect, this detector molecule is modified. This modification can include the addition of a template molecule that serves as a template for replication facilitated by polymerase activity. In a further aspect of this embodiment, the template molecule is a polynucleotide. In one aspect, the polymerase is a DNA polymerase or a RNA polymerase. In a particular aspect, the polymerase is Q-Amp. In a particular aspect, the detector molecule is an antibody specific for a particular target molecule. In a still further aspect, the target molecule is a prion protein or fragment thereof.

Another embodiment is directed to a kit for performing the methods of the present invention. The kit comprises an affinity ligand specific for and having affinity to a target molecule. In one aspect, the affinity ligand has a template conjugated to it, wherein the template is a nucleic acid molecule. The kit further comprises a replicase activity. In one aspect, the replicase is Q-Amp.

For a better understanding of the present invention, together with other and further objects thereof, reference is made to the accompanying drawings and detailed description and its scope will be pointed out in the appended claims.

### BRIEF DESCRIPTION OF THE DRAWINGS

FIG. 1 depicts the basic concept of the present invention;

FIG. 2 depicts the use of DNA templates in the present invention;

FIG. 3 depicts the use of RNA templates in the present invention;

FIG. 4 depicts one embodiment of the present invention.;

FIG. 5 depicts coating of a vesicle with antibody;

FIG. 6 depicts incubation with a detection antibody;

FIG. 7 depicts the results when Q-beta replicase is added;

FIG. 8 depicts an ELISA reaction; and

FIG. 9 depicts the reaction product from an ELISA -Q-Amp protocol.

### DETAILED DESCRIPTION OF THE INVENTION

The present invention pertains to methods of detecting molecules from a complex sample using a modified detector molecule. In particular, the modified detector molecule is specific for a particular target molecule and comprises a template that can be employed to amplify a signal thus indicating the presence of the target molecule.

One embodiment of the invention is directed to a method for detecting the presence or absence of a target molecule. The methods of the present invention comprise a detector molecule having affinity for the target molecule. In one aspect, this detector molecule is modified. This modification includes the conjugation of a template molecule that can serve as a template for replication facilitated by polymerase activity. In a further aspect of this embodiment, the template molecule is a polynucleotide. The template can be DNA, RNA, or modifications thereof. In one aspect, the polymerase is a DNA polymerase or a RNA polymerase. In a particular aspect, the polymerase is Q-Amp. In a particular aspect, the detector molecule is a modified antibody specific for a particular target molecule.

As used herein, the term “affinity” means exhibiting an attraction or capacity for binding. A specific affinity is an attraction that is directed to a particular feature or sequence of a molecule.

As used herein, the terms “polynucleotide” and “oligonucleotide” are used interchangeably, and include polymeric forms of nucleotides of any length, either deoxyribonucleotides or ribonucleotides, or analogs thereof. Polynucleotides can have any three-dimensional structure, and can perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be inputted into databases in a computer having a central processing unit and used for bioinformatics applications such as functional genomics and homology searching.

The term “protein” includes a compound of two or more subunit amino acids, amino acid analogs, peptides, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

As used herein, “fragment” refers to a molecule that originates from a parent polymeric molecule. A fragment is a processed molecule that can serve partially of completely as the parent polymeric molecule. It shares many of the features that are characteristic of the parent.

As used herein, “derivative” refers to a molecule that has been modified in some physiochemical fashion as compared to the parent molecule. For example, chemical groups can be added (or eliminated) to the parent molecule thus rendering a derivative of the parent.

The target molecule of the present invention includes proteins (peptides, etc.), nucleic acids, lipids, and combinations thereof. The target molecule needs to have sufficient complexity to allow it to interact with components of an immune system, such as an antibody. In one aspect of the present invention, the target molecule is a prion protein. In a particular aspect, the prion protein (“PrP”) is the pathological scrapies form of the protein (“PrP^{sc}”).

Transmissible spongiform encephalopathies (TSEs) are neurodegenerative infectious diseases that affect the central nervous system (CNS). TSEs include scrapies in sheep, Bovine Spongiform Encephalopathy (BSE) in cattle and Cruetzfeld-Jakob Disease (CJD), Guerstmann-Sträussler-Scheinker Syndrome (GSS), kuru, and Fatal Familial Insomnia (FFI) disease in humans. Common to all of these fatal diseases are long incubation periods and the accumulation of amyloid-like rods or scrapie associated fibrils (SAFs). The formation of SAFs is the result of extensive fibrillation of PrP^{SC}, the isoform of the endogenous and innocuous PrP^{C} protein (cellular protein—physiologically normal) that is associated with infectivity. The structural transformation of the soluble PrP^{C} to the insoluble PrP^{Sc} isoform marks the onset and progression to clinical prion disease.

The gene that encodes PrP^{C} is highly conserved and constitutively expressed from the prnP locus as a 35 kDA glycoprotein (Chesebro et al., 1985 Nature 315:331-33; Oesch et al., 1985 Cell 40:735-46; the entire teachings of which are incorporated herein by reference). Approximately one half of translated PrP^{C} is processed to the extracellular membrane where it is anchored to the plasma membrane by a C-terminal glycosyl-phophatidyl-inositol (GPI) anchor. However, PrP^{C} has also be found in two trans-membrane forms, one with the N-terminus inside the ER lumen (PrP^{Ntm}; 40-50%) and the other in the opposite orientation with the C-terminus inside the ER lumen (PrP^{Ctm}; 10%). It is unknown if these processing differences reflect the functional properties of these PrP^{C} forms.

PrP proteins have interesting structural characteristics, particularly the extraordinary transformation from its native, wildtype conformation PrP^{C} to the infectious PrP^{Sc}. The alteration in protein structure is marked by a transition from alpha-helix rich of PrP^{C} into beta-sheet rich regions in the C-terminal domain of the isoform associated with infectivity (Pan et al., 1993 PNAS, USA 90:10962-66; the entire teachings of which are incorporated herein by reference). Several biochemical traits distinguish the isoforms such as the insolubility of PrP^{Sc} in physiologic solutions and the resistance of its C-terminal domains (amino acids 90-231) to digestion by proteinase K. The structure of the non-protease treated full-length N-terminus of PrP is very flexible and without a single, stable structure based on NMR structural studies. Therefore, this region of PrP is most likely indistinguishable between the cellular and scrapie isoforms.

Binding of PrP to nucleic acids has been demonstrated many times through the observation of direct complex formation in vitro with purified protein and by copurification of nucleic acids from scrapie associated fibrils (SAFs) removed from infected tissue (Merz et al., 1981 Acta Neuropathol (Berl) 54(1):63-74; the entire teachings of which are incorporated herein by reference). It is this binding that may be responsible for converting the cellular form to the pathological scrapies form. For example, several thousand bases of the viral RNA genome of IAP were co-purified with SAF from infected tissue (Murdoch, et al., 1990 Virology 64(4): 1477-86; Akowitz et al., 1994 NAR 22(6):1101-07; the entire teachings of which are incorporated herein by reference). Such observations influenced studies to explore the possibility that nucleic acids were a required genetic component in the transmission of TSE, although no such genetic link has been experimentally determined to date.

Indirect evidence for an in vivo association between PrP and viral components is the observation that the rate of PrP^{Sc} formation is accelerated in cells affected with moloney murine leukemia virus (Carp et al., 1999 J Gen Virol 80(Pt 1):5-10; the entire teachings of which are incorporated herein by reference). There is further evidence of interactions between PrP and viral nucleic acids derived from in vitro studies that used recombinant, mammalian PrP proteins expressed in E.coli. Syrian Golden Hamster recombinant PrP^{C} (srPrP) has a surprising homology of in vitro activities with the nucleocapsid protein from HIV (Ncp7) (Tanchou et al., 1995 J Mol Biol 252:563-71; Gabus et al., 2001a J Mol Biol 307(4):1011-21; Gabus et al., 2001b J Biol Chem 276(22):19301-9; the entire teachings of which are incorporated herein by reference). srPrP has virtually the same level of activity as Ncp7 in the processes of DNA strand-transfer, nucleic acid chaperoning, HIV-RT priming, and the formation of condensed protein/nucleic acid structures. Double stranded DNA also induced the formation of similar condensed PrP structures, as well as resistance to proteinase K digestion (Nandi, 1998 Arch Virol 143(7):1251-63; Nandi and Leclerc, 1999 Arch Virol 144(9):1751-63; Nandi and Sizaret, 2001 Arch Virol 146:327-45; the entire teachings of which are incorporated herein by reference). Recently, two small RNA aptamers have been isolated based on their ability to bind to PrP proteins. One aptamer, AP1 (29 nt), was isolated using recombinant srPrP, and is predicted to fold into a compact structure containing three stacked G-quartets, a structure suggested to be important in binding to srPrP (Weiss et al., 1997 J Virol 71(11):8790-97; the entire teachings of which are incorporated herein by reference). Using a series of srPrP truncations, the authors localized the binding of AP1 to the flexible N-terminus, within amino acids 23-39.

The methods of the present invention can be used to detect the presence or absence of a target molecule using modified detector molecules. In one aspect, the modified detector molecule is an antibody. The antibody population can be either monoclonal antibodies, polyclonal antibodies, or a combination thereof. In one aspect, the antibody has affinity with PrP^{Sc}.

A monoclonal antibody (mAb) according to the present invention is intended to bind to, detect and qualitatively and quantitatively measure the presence of epitopes of prion proteins whether they are in soluble or insoluble form in various tissue specimens such as homogeneous or sections of brain, spleen, tonsils, white blood cells others and body fluids such as blood, cerebrospinal fluid saliva, urine or others. The present mABs bind to eptiopes of amino acids in a row or to epitopes of amino acids on different loops of the three-dimensional structure of native PrPs which are spatially close to each other. A particular group of the present antibodies binds only to native disease-specific PrP and not to native normal PrP.

The term monoclonal antibody comprises also chimeric monoclonal antibodies having similar properties, which are derived from different animals, such as human/mouse chimeric antibodies or any other chimeric molecule comprising the antigen-binding part of the monoclonal antibody (idiotype) with other molecules such as antibody fragments of other monoclonal antibodies or enzymes. See, U.S. Pat. No. 6,750,324, the entire teaching of which is incorporated herein by reference.

A fragment of a monoclonal antibody comprising the binding part of the monoclonal antibody (idiotype) likewise capable of specifically binding the antigen and is termed Fab or (Fab′)₂ depending on whether the monoclonal antibody is digested with papain or pepsin, respectively.

A synthetic antibody or fragments thereof designed according to the amino acids or substituted homologous amino acids composing the idiotype responsible for binding the antigen. Homologous amino acids are defined as exchanges within the following five groups. (1) Small aliphatic, nonpolar or slightly poor residues: alanine, serine, threonine, glycine, proline; (2) Polar, negatively charged residues and their amides: aspartic acid, asparagine, glutamic acid, glutamine; (3) Polar, positively charged residues: histidine, arginine, lysine; (4) Large aliphatic, nonpolar residues: methionine, leucine, isoleucine, valine, cysteine; (5) Large aromatic residues: phenylalanine, tyrosine, tryptophan. In one aspect the monoclonal antibodies of the present invention are 6H4, 34C9, 15B3 which are produced by hybridoma cell lines DSM ACC2295, DSM ACC2296 and DSM ACC2998, respectively. See, U.S. Pat. No. 6,765,088, the entire teaching of which is incorporated herein by reference.

The antibodies and fragments thereof are essential tools for immunological detection procedures based on the binding of the prion protein to the presented monoclonal antibodies in an antigen-antibody complex. The monoclonal antibodies of the present invention react with recombinant PrP as well as native or denatured PrP^{C} and PrP^{Sc} whether they are in soluble or insoluble state. The monoclonal antibodies react furtheron with PrP from different species, for example, humans, hamsters, pigs, sheep, cattle and mice. Furthermore, the present antibodies by forming an antigen-antibody complex between the presented monoclonal antibodies and the prion protein can be used to inhibit neurotoxic and infectious properties of the disease-specific prion protein.

The current invention relates to anti-idiotype antibodies which are antibodies that bind with their binding region (idiotype) to the binding region of the original monoclonal antibody. The anti-idiotype antibody mimics features of the original antigen, in this case features of PrP. Anti-idiotype antibodies are raised as polyclonal antibodies (serum) or monoclonal antibodies from animals immunized with the antibodies according to the invention. Anti-idiotype antibodies are valuable tools in detecting and blocking interactions of the original antigen (PrP), particularly interactions with receptors and can therefore be used in prevention and therapy of prion diseases.

A stable hybridoma cell line according to the present invention is capable of producing a monoclonal antibody as defined above over a prolonged time period of at least six months. Such cell lines are derived from the fusion of a spleen cell expressing the antibody derived from mice lacking a functional PrP gene, and a myeloma cell of mice providing survival of the fused cell lines using methods well known to those skilled in the art.

Suitable examples of hybridoma cell lines are DSM ATC2295, DSM ACC2296 and DSM ACC2298. The first two cell lines were deposited under the Budapest Treaty on Feb. 6, 1997 at the Deutsch Sammlung von Mikroorganisnien und Zellkulturen GmbH, Mascheroder Weg 1b, D-38124 Braunschweig, a recognized public depository for strains of microorganisms. The hybridome cell line producing mAB 15B3 was deposited Feb. 13, 1997 under number DSM ACCC2298 at the same depository.

The present method for the production of an antibody according to the invention comprises culturing a hybridoma cell line as mentioned above and isolating the monoclonal antibody from the supernatant of the growth media.

Culturing is carried out in a flasks in HT-medium or in a cell culturing system called “technomouse” in serum-free, synthetic medium (Turbodoma medium, supplied by Messi, Zurich). In a “technomouse” hybridoma cells are cultured in a sterile chamber surrounded by a protein-impermeable membrane that is perfused by the respective medium in a constant flow rate (for example, turbomedium at 80 ml/h); antibodies are collected from the chamber with the help of a syringe at regular intervals.

Isolation of monoclonal antibodies is carried out by extraction from the supernatant by conventional biochemical methods, e.g., by use of affinity columns with the corresponding immobilized antigen or by any other method used in the art, such as gel filtration or ion exchange chromatography. In the “technomouse” supplied with serum-free turbomedium antibody concentrations and purities are achieved that need no further extracting procedures.

Chimeric antibodies and fragments thereof can be produced by genetic engineering methods, e.g., by sequencing the antibody or the desired fragment thereof and constructing DNAs coding for the chimeric antibody or the fragment thereof which DNAs are inserted into an appropriate expression vector and expressed to produce the antibody or the fragment thereof in both procaryotic or eukaryotic cell lines.

A fragment binding to a PrP epitope can be combined with a human heavy chain to produce chimeric antibodies for use in humans as therapeutic or preventive agents against a prion disease. A fragment binding to a PrP epitope can also be combined with other enzymes, proteins or molecules to give rise to chimeric molecules combining the biological functions of these, e.g., for targeting an enzymatic activity to a place defined by the proximity of the PrP epitope.

The present method for the production of a hybridoma cell line comprises administering to PrP^{0/0} mice (knockout mice without a functional PrP gene) an immunizing amount of a recombinant pure prion protein PrP, removing the spleen from the immunized mice, recovering splenocytes therefrom, fusing the latter with appropriate myeloma cells, growing the cells in a selection medium which does not support survival of the unfused cells, e.g., in HAT medium, screening the supernatants of the surviving hybridoma cells with recombinant PrP for the presence of antibodies to detect recombinant bovine PrP by an ELISA procedure and to detect native bovine PrP^{Sc} by a conformation-sensitive ELIFA procedure and isolating the positive cells. Positive hybridomas were selected and cloned twice by the limiting dilution method before the antibody was characterized and the epitope was mapped on a peptide library.

One peptide library that can be used is commercially available from Jerini Biotools (Berlin Germany). It consists of 104 spots with peptides of 13 amino acids, whereby the sequence of each peptide overlaps with 11 amino acids of the foregoing peptide.

An immunizing amount of a recombinant bovine prion protein is from about 50 to 100 μg. It is administered dissolved in an appropriate solvent, e.g., PBS and Freund's adjuvant several times, e.g., three times, subcutaneously followed by an intraperitoneal and an intravenous injection ultimately prior to spleen removal.

Appropriate myeloma cells are for example P3X63Ag8U.1 and are deposited and available under ATCC CRL 1597.

Recovering spleen cells and fusion conditions follow standard procedures, for example, as described by Kennett (Kennett, R. H. Fusion centriugation of cells suspended in polyethylene glycol. In Monoclonal antibodies. Hybridomas: a new dimension in biological analysis. (New York: Plenum Press) (1980) pp. 365-367).

An immunological detection of prion disease, especially disease-specific PrP^{Sc} protein in a sample comprising, e.g., biological material of an animal or human is detected, comprises incubating a suitable affinity ligand, e.g., an appropriate antibody, with the sample under condition suitable for forming an antibody-prion protein complex which conditions are well known to those skilled in the art. In one aspect, a specific monoclonal antibody according to the invention is able to detect PrP^{Sc} without prior protease-digestion of the tissue specimen to be examined. In one aspect, the affinity ligand, antibody, is modified in that it has conjugated to it a template.

The biological material can be insoluble or soluble in buffer or body fluids. It can be derived from any part of the body, e. g., from the brain or the tissue sections, in which case it is used in form of a homogenate, or any body fluid, e. g., cerebrospinal fluid, urine, saliva or blood. In the case of body fluids, fluid-resident cells, e.g., white blood cells in the case of blood expressing PrP can be purified and analyzed either in immunohistochemistry or as a homogenate.

Tissue homogenates and body fluids include, e.g., biopsy of brain, lymph nodes, spleens, tonsils, peripheral nerves, cerebrospinal fluids, urine, platelets or white blood cells.

The template, e.g., a polynucleotide (either RNA or DNA, including modifications thereof), can be conjugated to an antibody using, e.g., biotin. In this scenario, the affinity ligand, antibody, comprises avidin which provides a receptor site for biotin. This method of conjugation is well known to those skilled in the art. Other suitable methods are also appreciated by the skilled artisan for conjugating a nucleic acid molecule onto an antibody.

Suitable templates for the present method include both RNA and DNA nucleic acids, including modifications made thereto. The template of the present invention is a polynucleotide that can be received and amplified by Q-beta replicase, Q-Amp, and other nucleic acid replicases for DNA or RNA. The particular nucleotide sequence of the template is not important. However, it must be amenable to replication by, for example, Q-Amp.

Examples of suitable DNA templates are midi-varient DNA (MDV DNA), mini-varient DNA (MNV DNA), MNV-AP1 DNA, MNVUP DNA, MNVLO DNA, and combinations thereof.

The DNA sequence encoding MDV (SEQ ID NO 1) is:

5′ GGGGACCCCCCGGAAGGGGGGACGAGGTGCGGGCACCTCGTACGGGA
GTTCGACCGTGACGAGTCACGGGCTAGCGCTTTCGCGCTCTCCCAGGTGA
CGCCTCGTGAAGAGGCGCGACCTTCGTGCGTTTCGGCGACGCACGAGAAC
CGCCACGCTGCTTCGCAGCGTGGCCCCTTCGCGCAGCCCGCTGCGCGAGG
TGACCCCCGAAGGGGGGTTCCCCA 3′

The DNA sequence encoding MNV (SEQ ID NO 2) is:

5′ GGGTTCATAGCCTATTCGGCTTTTAAAGGACCTTTTTCCCTCGCGTA
GCTAGCTACGCGAGGTGAC CCCCCGAAGGGGGGTGCCCC 3′

The DNA sequence encoding MNV-AP1 (SEQ ID NO 3) is:

5′ GGGTTCATAGCCTATTCGGCTTCGCGCATGGGAATTTGAGGGACGAT
GGGGAAGTGGGAGCGCGTTTTAAAGGACCTTTTTCCCTCGCGTAGCTAGC
TACGCGAGGTGACCCCCCGAAGGGGGGTGCCCC 3′

The DNA sequence encoding MNVUP (SEQ ID NO 4) is:

5′ GGGTTCATAGCCTATTCGGCTTCGCGCCCGTTTATAATACTTAGTGA
GCGCGTTTTAAAGGACCTTTTTCCCTCGCGTAGCTAGCTACGCGAGGTGA
CCCCCCGAAGGGGGGTGCCCC 3′

The DNA sequence encoding MNVLO (SEQ ID NO 5) is:

5′ GGGTTCATAGCCTATTCGGCTTCGCGCCCCTGGGGTTTGCCTCAGGA
GCGCGTTTTAAAGGACCTTTTTCCCTTGCGTAGCTAGCTACGCGAGGTGA
CCCCCCGAAGGGGGGTGCCCC 3′

In one aspect of the present invention, the template is an RNA molecule. For example, RQ 11+12. The RNA sequence for RQ11+12 (SEQ ID NO 6) is:

5′ GGGGUUUCCAACCGGAAUUUGAGGGAUGCCUAGGCAUCCCCCGUGCG
UCCCUUUACGAGGGAUUGUCGACUCUAGUCGACGUCUGGGCGAAAAAUGU
ACGAGAGGACCUUUUCGGUACAGACGGUACCUGAGGGAUGCCUAGGCAUC
CCCCGCGCCGGUUUCGGACCUCCAGUGCGUGUUACCGCACUGUCGACCC
3′

There is no absolute length requirement for participating polynucleotide sequences. However, in one aspect, the range is from about 20 to about 10,000 nucleotides. One of ordinary skill in the art will be able to determine the appropriate length of nucleotide sequence to employ for the present invention.

It is understood that complementary base-pairing of individual base pairs generally follows Chargaff's Rule wherein an adenine pairs with an uracil (or thymine if DNA) and guanine pairs with cytosine. However, there are modified bases that account for unconventional base-pairing. A modified nucleic acid is understood to mean herein a DNA or RNA nucleic acid molecule that contains chemically modified nucleotides. The term “nucleic acid analogue” is understood herein to denote non-nucleic acid molecules such as “PNA” and morpholino that can engage in base-pairing interactions with conventional nucleic acids. These modified bases and nucleic acid analogues are considered to be within the scope of the instant invention. For example, nucleotides containing deazaguaine and uracil bases can be used in place of guanine and thymine, respectively, to decrease the thermal stability of probes. Similarly, 5-methyl-cytosine can be substituted for cytosine in complexes if increased thermal stability is desired. Modification to the sugar moiety can also occur and is embraced by the present invention. For example, modification to the ribose sugar moiety through the addition of 2′-O-methyl groups which can be used to reduce the nuclease susceptibility of RNA molecules. Modifications occurring with different moieties of the nucleic acid backbone are also within the scope of this invention. For example, the use of methyl phosphate, methyl phosphonate or phosphorothioate linkages to remove negative charges from the phosphodiesters backbone can be used.

As mentioned above, the templates can be amplified using Q-Amp. Q-Amp is derived from Q-beta replicase. Q-beta replicase can be isolated and purified from Q-beta bacteriophage. Q-Amp is derived from the Q-beta replicase and comprises eukaryotic elongation factor Ts (Ef-Ts), eukaryotic elongation factor Tu (Ef-Tu), S1 nuclease, and a Replicase component. (Q-Amp is available from Q-RNA, Inc, New York, N.Y.) Q-Amp recognizes nucleic acid templates and can amplify them exponentially, e.g., up to one billion-fold, in fifteen minutes under isothermal conditions. Templates for Q-Amp can contain sequence insertions that may have specific functional applications. It should be appreciated by those skilled in the art, that replicases other than Q-Amp and Q-beta replicase may be used and are considered to be within the scope of this invention.

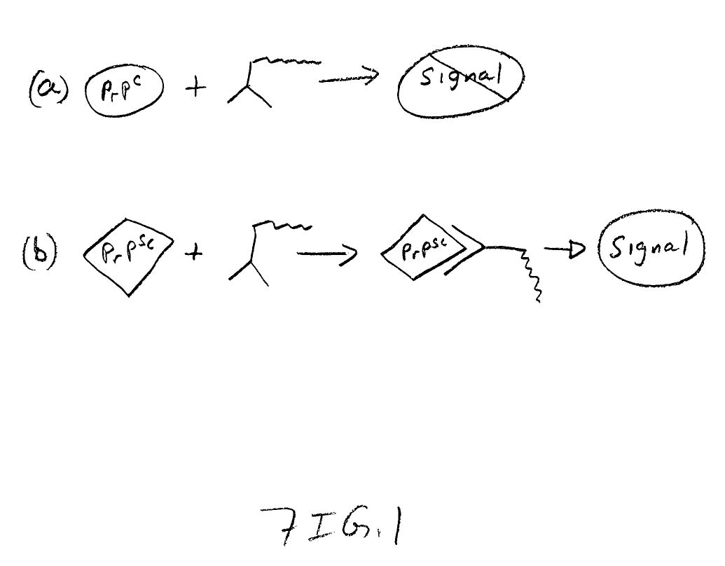

FIG. 1 illustrates the principle underlying the methods of the present invention. In FIG. 1a, native prion protein (PrP^{C}) is admixed with an antibody specific for the scrapies form of prion (PrP^{Sc}). The antibody has conjugated to it a template represented by the squiggly line. As depicted in FIG. 1a, the antibody does not react with or bind to the native prion, hence there will be no antibody-antigen complex resulting in no signal. However, FIG. 1b depicts a scrapies form prion admixed with an antibody specific for PrP^{Sc}. A complex is formed resulting in the formation of a signal.

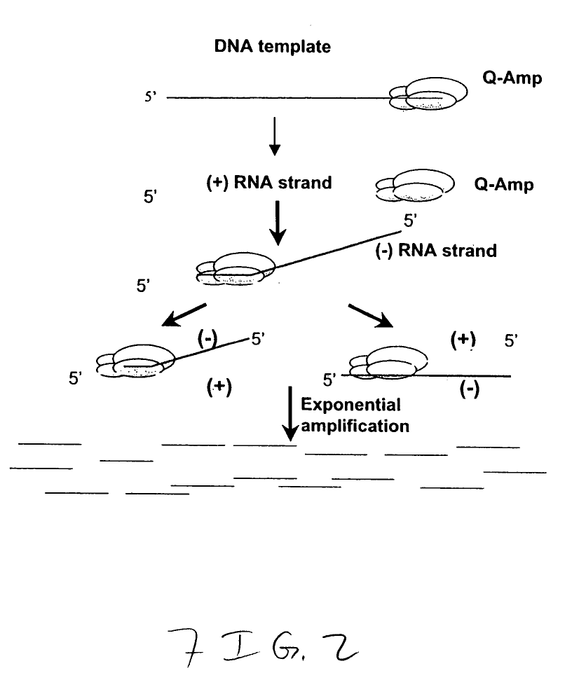

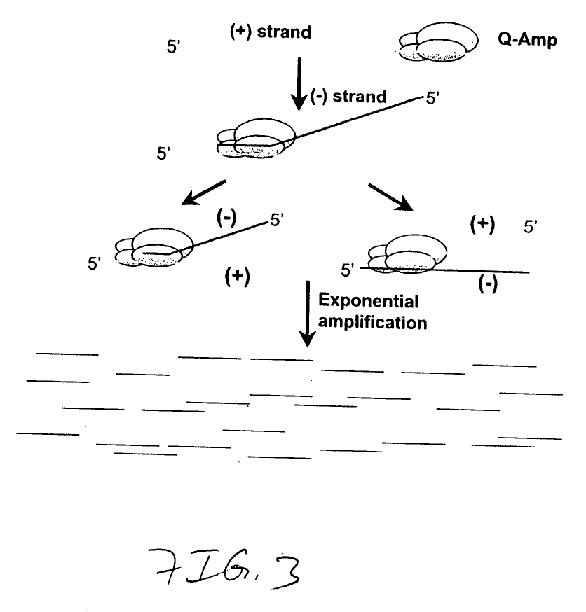

Detection of an antibody-antigen complex can be accomplished using several techniques. In the present invention, detection of an antibody-antigen complex is indicative of the presence of one or more target molecules within a given sample. In one aspect of the present invention, the template facilitates a detectable signal. As indicated above, the antibody of the present invention is modified comprising one or more template molecules. These templates are substrates for Q-Amp and are replicated under suitable conditions which are known to those skilled in the art. As mentioned above, Q-Amp can amplify a template a billion-fold plus in a matter of minutes. For example, if the template is a DNA molecule (like one described above), then Q-Amp can amplify this DNA template a billion-fold plus in a short period of time. See FIG. 2. The nascent DNA single-strands will comprise both (−) and (+) strands that under proper conditions the complementary strands will hybridize forming double-stranded DNA molecules. These newly synthesized double-stranded DNA molecules can be detected using, e.g., an intercalating agent such as ethydium bromide which will emit a signal when intercalated within a double-stranded DNA. Other classes of nucleotides can serve as templates such as RNA. See FIG. 3.

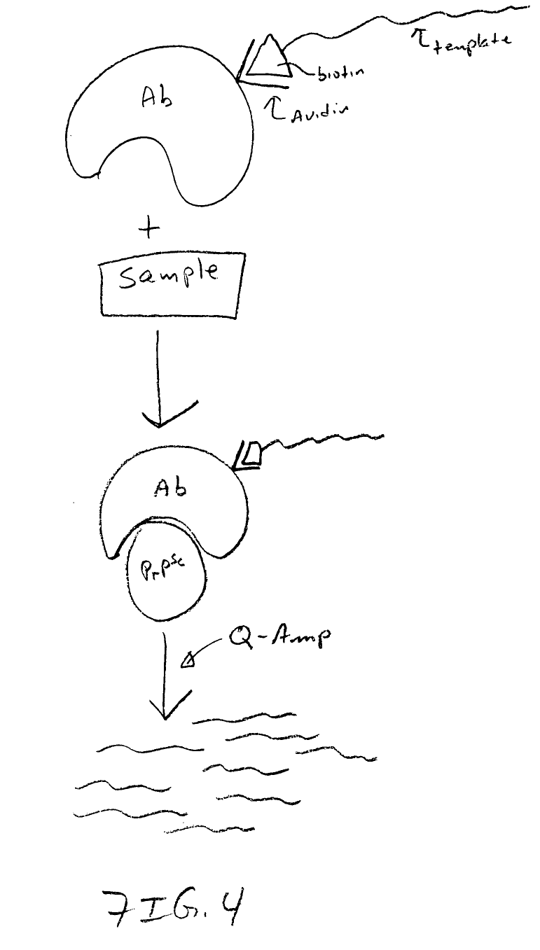

FIG. 4 depicts one embodiment of the present invention. This embodiment includes an antibody specific for PrP^{Sc} which is conjugated to a template via an avidin-biotin interaction. The conjugated antibody is admixed with a sample putatively containing PrP^{Sc}. The conditions are suitable for antibody-antigen complex formation. The complex is separated from free antibody. Q-Amp is admixed with isolated complex to form nascent nucleic acid molecules. These nascent molecules can be detected using methods described herein as well as those well known to those skilled in the art.

In order to minimize or eliminate false positives, the antibody-antigen complex has to be isolated from the free (unbound) antibody. There are several well accepted methods for effectuating this isolation. For example, isolation can be realized by employing a size separation technique. The target (antigen) can be fixed to a substrate. Following a suitable period of incubation with a homogenous or heterogenous preparation of template containing antibody, a wash step can be performed eliminating unbound antibody. Thus, the remaining antibody is that which is bound to the target.

One skilled in the art will appreciate that there are other well known methods for producing a signal such as the use of a label that emits energy which can be detected by a suitable detector. For example, labeled nucleotide base precursors can be used in the replication reaction. For example, adenine can be labeled with a radioactive label or a fluorescence label, or any other suitable label that can be detected. As the nascent nucleic acid is being synthesized using the conjugated template, labeled nucleotides will be incorporated into the nascent nucleotide.

In one embodiment, a kit is describe for performing the methods of the present invention. The kit comprises an affinity ligand specific for and having affinity to a target molecule. In one aspect, the affinity ligand is an antibody. In a particular aspect, the affinity ligand has conjugated to it a template molecule. The template can be a nucleic acid molecule. Specifically, the template can be DNA, RNA, or a modification thereof. Examples of a suitable template are MDV, MNV, MNV-AP1, MNVUP, MNVLO, RQ11+12, fragments and derivatives thereof. Appropriate reagents such as buffers known to those skilled in the art can be supplied in the kit as well. The kit also comprises a replicase activity. An example of a suitable replicase is Q-Amp. Instructions can also be included within the kit.

### EXAMPLE

Immunoglobulin ELISA-Qamp Protocol

I. Coat with Capture Antibody:

1. Dilute the purified mouse IgG, Whole Molecule (Pierce, cat. no. 31204), to 334 fm/ml (2 μg/ml, 10 fm/30 μl) in coating buffer (PBS or 0.1 M NaHCO3, pH 8.6).

and to prepare 10× serial dilutions of mouse IgG (from 10 fM/30 μl to 0.1 aM/30 μl).

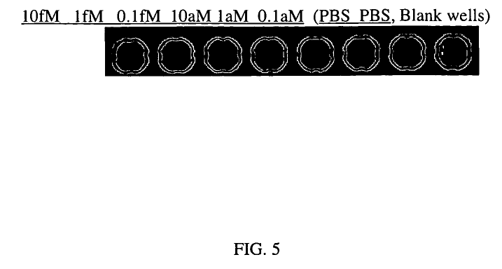

2.Add 30 μl per well to an enhanced protein-binding ELISA strips (e.g., Nunc TopYieid Strips, cat. no. 24-8909). See FIG. 5.

3. Shake plate to ensure all wells are covered by capture antibody solution.

4 Seal plate to prevent evaporation. and incubate for overnight at 4° C. ( or 1 hour at 37° C.).

5. 3. Bring the plate to RT, remove the capture antibody solution. Wash the plate 3× with PBS/Tween*. For each wash, wells are filled with 200 μl PBS/Tween and allowed to stand at least 1 minute prior to aspirating.

II. Blocking:

1. Block the plate with 200 μl blocking buffer* per well.

2. Cover the plate and incubate for overnight at 4° C. or at room temperature for 60 minutes.

3. Wash the plate 3× with PBS/Tween, as in Section I, Step 4, of this protocol.

III. Incubation with Detection Antibody:

1. Dilute biotinylated anti-mouse IgG (Pierce, cat. no. 31800) in 1:10 blocking buffer to 0.05-0.20 μg/ml (10-40 fm per 30 μl)

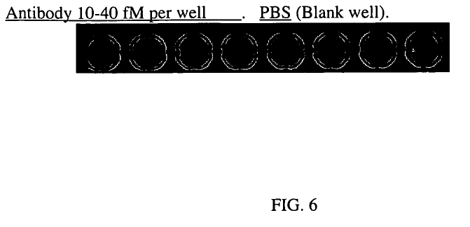

2. Add 30 μl per well. See FIG. 6.

3. Cover the plate and incubate at room temperature for 1 hour.

4. Wash the plate 6× with PBS/Tween,

IV. Add Neuroavidin-BiotinDNA-MNV11 Mix):

1. Dilute BiotinDNA-MNV11 (stock solution at 5 pM/μl) to 0.2pM/μl in 10 mM tris.HCl, pH 7.4, 150 mM NaCl. (100 μl)

2. Dilute Neuroavidin to 0.2 pM/μl in 10 mM tris.HCl, pH 7.4, 150 mM NaCl. (100 μl)

3. Mix and vortex. Incubate at RT 30-60 min. Solution may be stored for several weeks at 4 C.

4. Dilute Neuroavidin-BiotinDNA-MNV11 mix to 2-10 fM/30 ml in 1:10 blocking buffer. Add 30 μl per well.

5. Cover the plate and incubate at room temperature for 60 minutes.

6 Wash the plate 8× with PBS/Tween, 2× with H2O.

V. Add Qbeta Replicase Mix and Develop.

1. Dilute Qbeta replicase to 0.09-0.18 mg/ml (buffer 1×G, 0.5 mM NTP, 1.5 μg propidium iodide/ml).

2. Add 30 μl per well.

3. Incubate at 30-37 C (20-30 min).

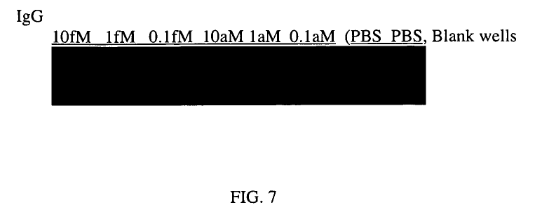

4. Transfer to UV box. Take a digital picture (yellow filter), See FIG. 7. High backgrounds in blank wells or poor consistency of replicates can be overcome by increasing the stringency of washes and optimizing the concentration of detection antibody. For example, during washes, the wells can be soaked for ˜1 minute intervals. Moreover, lower concentrations of detecting antibody and bioDNAMNV11 or more washes can reduce background

| | | |
|---|---|---|
| Reagents: | Tris-HCl, pH 7.4, | 1 M |
| | MgCl_{2,} | 1 M |
| | DTT, | 0.1 M |
| | NTP | 25 mM |
| | Propidium | 0.1 μg/μl |
| | Qbeta replicase | 4 mg/ml |
| | H₂O/Sigma | |
| Buffer 5xG: | (1 ml): | 400μ Tris-HCl |
| | | 50 μl MgCl₂ |
| | | 50 μl DTT |
| | | 500 μl H₂O |

Solutions
Coating Buffer PBS Solution

PBS, pH 7.2-7.4 NaCl 80.0 g

Na2HPO4 11.6 g

PBS/Tween KH2PO4 2.0 g

PBS KCl 2.0 g

Tween-20 0.05% ddH2O to 10 L

Adjust pH to 7.2-7.4

Blocking Buffer

PBS

Nonfat dry milk 5%-10%, DNA herring sperm (SIGMA, cat. D3159-10 G) 10 mg/ml.

High backgrounds in blank wells or poor consistency of replicates can be overcome by increasing the stringency of washes and optimizing the concentration of detection antibody and avidin-biotinDNA. For example, during washes, the wells can be soaked for ˜2-5 minute intervals. Moreover, lower concentrations of detecting antibody or more washes after the avidin-biotinDNA stage can reduce background.

The comparison of the sensitivity of the standard ELISA detection and ELISA-Qamp.

I. Standard ELISA Protocol.

1. Mouse IgG 40 fM

3. Neuroavidin_Biotin-Horseradish Peroxidase (1:50000) mix (1:1) (50 μl).

4. Detection: Turbo TMB-ELISA (50 μl), 20 min.

2. Biotinylated anti-Mouse IgG antibody.

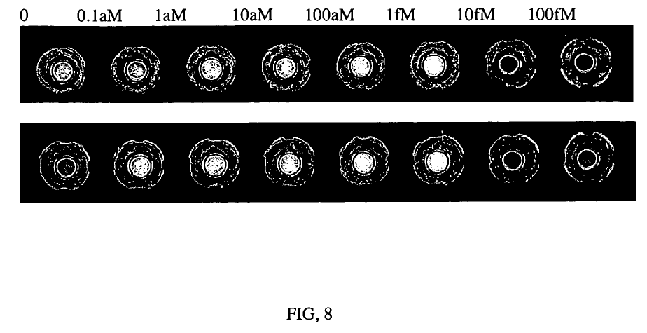

See FIG. 8.

II. ELISA-Qamp Protocol.

1. Mouse IgG 40 fM

3. Neuroavidin_biotin-dMNV11 (1:1), 4 fM (30 μl)

4. Detection: Qbeta replicase (Qbeta repl. 2.4 μg/30 μl. 0.75 mM NTP, 20 mM Mg++. 15 min

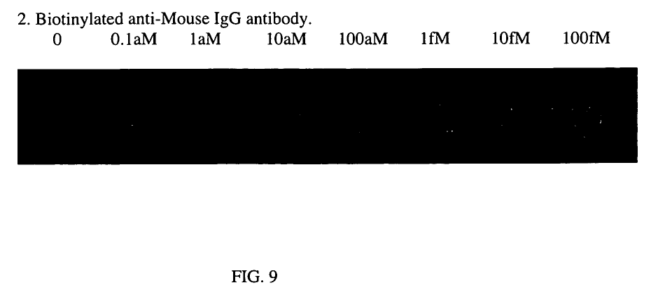

See FIG. 9.

Although the invention has been described with respect to various embodiments, it should be realized this invention is also capable of a wide variety of further and other embodiments within the spirit and scope of the appended claims.

1. A method for detecting the presence or absence of a target molecule in a sample, comprising:
(a) admixing an antibody specific for said target molecule with said sample under conditions suitable for forming an antibody-antigen complex, wherein said antibody has conjugated to it a template, wherein said antigen is said target molecule;
(b) admixing a replicase to said complex of (a);and
(c) detecting the presence or absence of said antibody-antigen complex.

2. The method of claim 1, wherein said target molecule is PrP or a derivative thereof.

3. The method of claim 2, wherein said PrP is PrP^{Sc}.

4. The method of claim 1, wherein said sample comprising animal tissue.

5. The method of claim 4, wherein said animal is human.

6. The method of claim 4, wherein said tissue is selected from the group consisting of cerebrospinal fluid, urine, saliva, blood, brain, lymph node, spleen tonsils, peripheral nerves, platelets, white blood cells and a combination thereof.

7. The method of claim 1, wherein said antibody is a monoclonal antibody, polyclonal antibody, or a combination thereof.

8. The method of claim 7, wherein said antibody is a monoclonal antibody.

9. The method of claim 8, wherein said monoclonal antibody is selected from the group consisting of 6H4, 34C9, 15B3 and a combination thereof.

10. The method of claim 1, wherein said template is a nucleic acid molecule.

11. The method of claim 10, wherein said nucleic acid molecule is DNA, RNA or modifications thereof.

12. The method of claim 10, wherein said template is selected from the group consisting of MDV, MNV, MNV-AP1, MNVUP, MNVLO, RQ 11+12, fragments and derivatives thereof.

13. The method of claim 1, wherein said replicase is Q-Amp.

14. The method of claim 1 further comprising isolating said antibody-antigen complex from free antibody.

15. The method of claim 14, wherein isolation is accomplished by size exclusion fractionation.

16. A kit for determining the presence or absence of a target molecule in a sample, comprising:
(a) an affinity ligand having an affinity for said target molecule, wherein said affinity ligand has conjugated to it a template molecule; and
(b) a replicase capable of replicating said template.

17. The kit of claim 16, wherein said affinity ligand is an antibody.

18. The kit of claim 17, wherein said antibody is selected from the group consisting of 6H4, 34C9, 15B3, and a combination thereof.

19. The kit of claim 16, wherein said target molecule is a PrP protein.

20. The kit of claim 19, wherein said PrP is PrP^{Sc}.

21. The kit of claim 16, wherein said replicase is Q-Amp.

22. The kit of claim 16, wherein said template is a nucleic acid.

23. The kit of claim 22, wherein said nucleic acid is DNA, RNA, or a modification thereof.

24. The kit of claim 22, wherein said template is selected from the group consisting of MDV, MNV, MNV-AP1, MNVUP, MNVLO, RQ11+12, fragments and derivatives thereof.
